(19) United States
(12) Patent Application Publication
(10) Pub. No.: US 2002/0173839 A1
(43) Pub. Date: Nov. 21, 2002

(54) Intravascular flow modifier and reinforcement device with connected segments

(76) Inventors: Eric W. Leopold, Redwood City, CA (US); Andrew J. DeNardo, Carmel, IN (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/122,257

(22) Filed: Apr. 12, 2002

Related U.S. Application Data
(63) Continuation-in-part of application No. 09/747,456, filed on Dec. 22, 2000, now Pat. No. 6,416,541.
Application No. 09/747,456 is a division of application No. 09/122,243, filed on Jul. 24, 1998, now Pat. No. 6,165,194.

(51) Int. Cl. A61F 2/06

(52) U.S. Cl. 623/1.15

(57) ABSTRACT

A stent includes a cylindrical frame and a plurality of connecting segments connecting opposed portions of the frame. The frame may be formed from either of a single loop of resilient wire formed into a series of arcuate sections and longitudinal connecting sections, two pieces of resilient wire each formed into a half-frame having a series of arcuate sections and longitudinal connecting sections or from a piece of laser cut hypotubing. For the resilient wire frames, the connecting segments may be either of a single metal or plastic band wrapped around opposed longitudinal sections, joined individual bands wrapped around opposed longitudinal sections, or a piece of solder joining opposed longitudinal sections. For the hypotubing frame, the connecting segments are pieces of remaining hypotubing joining opposed longitudinal sections.

### RELATED APPLICATIONS

This is a continuation-in-part of application Ser. No. 09/747,456, filed Dec. 22, 2000 which is a divisional of application Ser. No. 09/122,243 filed Jul. 24, 1998, now U.S. Pat. No. 6,165,194.

### BACKGROUND OF THE INVENTION

1. Field of the Invention

The present invention relates to a reinforcement device, i.e., stent, for use within a body vessel, and more particularly, to a stent for use in combination with vasoocclusive devices placed in an aneurysm for the purpose of occluding an aneurysm, that provides reinforcement for the area of the blood vessel in the vicinity of the aneurysm.

2. Description of the Related Art

The progress of the medical arts related to treatment of vascular malformations has dramatically improved with the availability of intravascular devices capable of operating entirely within the vasculature. Thus, many highly invasive surgical procedures and inoperable conditions have been treated by the use of an expanding number of devices and procedures designed for those purposes. One particularly useful development in the medical arts has been the ability to treat defects in relatively small arteries and veins, such as those in the neurovascular system, by use of an infusion catheter and the placement of embolic coils and the like in areas where the malformation is likely to cause or has already caused a rupture in the blood vessel. More specifically, it has been found that the treatment of aneurysms by such devices and procedures allows the medical practitioner to avoid otherwise risky medical procedures. For example, when the placement of the defect is in the brain, a great deal of difficulty is presented to treatment of small defects in the blood vessels with conventional surgical techniques. For these reasons, the progress in development of devices to treat such defects has been encouraged and has produced useful results in a wide variety of patients.

One aspect of these surgical treatments is that an aneurysm or other malformation is symptomatic of a general weakening of the vasculature in the area containing the aneurysm, and mere treatment of the aneurysm does not necessarily prevent a subsequent rupture in the surrounding area of the vessel. Moreover, it is often desirable to provide a means to prevent the migration of the vasoocclusive devices, such as coils and the like, out of the aneurysm in the event that the aneurysm has a relatively large neck to dome ratio.

Stents, which are tubular reinforcements inserted into a blood vessel to provide an open path within the blood vessel, have been widely used in intravascular angioplasty treatment of occluded cardiac arteries. In such a case, the stent is inserted after an angioplasty procedure or the like in order to prevent restenosis of the artery. In these applications, the stents are often deployed by use of inflatable balloons, or mechanical devices which force the stent open, thereby reinforcing the artery wall in the clear through-path in the center of the artery after the angioplasty procedure to prevent restenosis.

While such procedures may be useful in certain aspects of vascular surgery in which vasoocclusive devices are used, the weakness of the vasculature and the inaccessibility of the interior of the aneurysm from the vessel after the placement of such a stent, places limits on the applicability of such stents in procedures to repair aneurysms, particularly neuro-vascular aneurysms. Furthermore, the use of placement techniques, such as balloons or mechanical expansions of the type often found to be useful in cardiac surgery are relatively less useful in vasoocclusive surgery, particularly when tiny vessels, such as those found in the brain, are to be treated.

Hence, those skilled in the art have recognized a need for a stent compatible with techniques in vasoocclusive treatment of aneurysms that provides selective reinforcement in the vicinity of the artery, while avoiding any unnecessary trauma or risk of rupture to the blood vessel. The need for a stent with structural integrity that both allows for placement without a balloon or mechanical expansion and provides sufficient radial support when in a deployed state has also been recognized. The present invention provides these and other advantages.

### SUMMARY OF THE INVENTION

Briefly, and in general terms, the invention relates to various configurations of stents designed for use in the treatment of aneurysms and ischemic diseases.

In a first aspect, the invention relates to a stent for use in the intravascular treatment of blood vessels. The stent includes a generally cylindrical frame formed of an elongate resilient wire. The two free ends of the wire extend distally from the proximal end of the frame and transition at a first point to a pair of opposed first arcuate sections. Thereafter the frame transitions to a pair of opposed first longitudinal sections for a length to a second point and then transitions to a pair of opposed second arcuate sections and a pair of opposed second longitudinal sections. The frame proceeds similarly in this pattern to the distal end of the frame. The stent further includes a plurality of connecting segments which connect a plurality of opposed longitudinal sections.

In detailed aspects, the frame is formed from a material having properties that provide it with a predeployed essentially flat configuration and a deployed generally cylindrical configuration. In other detailed aspects, the connecting segments are located on both sides of the frame or alternatively only one side of the frame. In another detailed aspect, the connecting segments comprise a pair of bands. One of the bands is wrapped around one of a pair of opposed longitudinal sections. The first and second bands are secured together, thereby connecting the opposed longitudinal sections. In yet another detailed facet, the connecting segments comprise a single band secured around both of an opposed pair of longitudinal sections. In still another detailed facet, the connecting segments comprise a piece of solder spanning between a pair of opposed longitudinal sections.

In another aspect, the invention relates to a stent for use in the intravascular treatment of blood vessels that includes a first half-frame and a second half-frame. Each of the half-frames includes a plurality of arcuate sections connected by longitudinal sections. The stent further includes a plurality of connecting segments. These segments secure a plurality of first half-frame longitudinal sections to a plurality of second half-frame longitudinal sections such that the first half-frame and the second half-frame form a cylinder.

In a detailed aspect, the arcuate sections of the stent have a chevron configuration when viewed from a first direction and a bowed configuration when viewed from a second direction approximately 90° offset from the first direction. In further detailed aspects, the point of the chevron is directed toward the proximal end of the stent while the arcuate sections bow toward the proximal end of the stent. In still further detailed aspects, the connecting segments are located on only one side of the cylinder or alternatively on both sides of the cylinder.

In another detailed facet of the invention, each of the first and second half-frames are formed from a piece of elongate resilient wire with a first end extending distally from the proximal end of the half frame. The wire transitions at a first point to a first arcuate section and then transitions to a first longitudinal section for a length to a second point. Thereafter the wire transitions to a second arcuate section and a second longitudinal section and proceeds similarly to the distal end of the half frame. In another detailed aspect of the invention, the first and second half-frames and the connecting segments are formed from a single piece of hypotubing with portions removed to form first and second half-frame patterns and the plurality of connecting segments. Each of the half frame has an alternating arcuate section—longitudinal section configuration as described above with respect to the wire configuration. With respect to both the wire configuration and the hypotubing configuration, each of the half frames may have a predeployed essentially flat configuration and a deployed generally cylindrical configuration and/or a predeployed radially compressed configuration and a deployed generally cylindrical configuration.

In another aspect, the invention relates to a stent for use in the intravascular treatment of blood vessels that includes a first half-frame and a second half-frame, each of which includes a plurality of arcuate loop sections which comprise a pair of arcuate sections connected at each end by a longitudinal connecting section. The stent also includes a plurality of connecting segments that secure a plurality of first half-frame arcuate loop sections to a plurality of second half-frame arcuate loop sections such that the first half-frame and the second half-frame form a cylinder.

In a detailed aspect, the first and second half-frames are formed from a material having properties that provide it with a predeployed radially compressed configuration and a deployed generally cylindrical configuration. In other detailed facets, the connecting segments are located on only one side of the cylinder or alternatively are located on both sides of the cylinder. In another detailed aspect the first and second half-frame arcuate loop sections are secured such that the first half-frame arcuate loop sections are longitudinally offset from the second half-frame arcuate loop sections.

The devices, systems and methods of the present invention provide important advantages over prior art devices in that they eliminate the necessity for balloon or mechanical placement devices which can cause unnecessary trauma to the delicate vasculature which has already been damaged by the presence of the aneurysm. For this reason, the invention is particularly useful to cover and reinforce large neck aneurysms. The presence of the longitudinal sections and the connecting segments improves the pushability of the stent, thereby enhancing the ability to deploy and place the stent within the vasculature, an issue of considerable importance if neither balloon nor mechanical placement methods are to be used. The connecting segments also increase the structural integrity of the stent and provide sufficient radial support when the stent is in a deployed state.

Another advantage of the present invention is that it maybe used in arteries up to renal size while still providing the benefits of placement without the use of balloons or mechanical expansions. One significant benefit in such an application is that the flow through the vessel is never fully occluded by the placement of the device in the invention, and it is possible to place the stent from a free flow guiding catheter that is relatively small in diameter compared to the inside diameter of the blood vessel being treated.

While certain features of the invention and its use have been described, it will be appreciated by those skilled in the art that many forms of the invention may be used for specific applications in the medical treatment of deformations of the vasculature. Other features and advantages of the present invention will become apparent from the following detailed description taken in conjunction with the accompanying drawings, which illustrate by way of example, the principles of the invention.

### BRIEF DESCRIPTION OF THE DRAWINGS

FIG. 1 is a perspective view of a stent in a deployed state and configured in accordance with one embodiment of the invention, having a frame formed from a single loop of wire formed into a series of arcuate sections and longitudinal connecting sections and a plurality of connecting segments connecting opposed longitudinal connecting sections;

FIG. 2 is a side view of the deployed stent of FIG. 1;

FIG. 3 is a plan view of the stent of FIG. 1 in a predeployed, flattened state;

FIG. 4 is a cross section of a guiding catheter revealing a plan view of the stent of FIG. 3 positioned within the catheter in a predeployed, flattened and compressed state;

FIG. 5 is a side view of a stent at a transition point between the predeployed state of FIGS. 3 and 4 and the deployed state of FIGS. 1 and 2;

FIG. 6 is a side view of a deployed stent illustrating an alternate configuration in which the arcuate sections of the stent are more densely located in the middle portion of the stent;

FIG. 7 is a plan view of a predeployed stent illustrating an alternate configuration in which the radii of the arcuate sections vary along the length of the stent;

FIG. 8 is an illustration of a mandrel upon which the stent of FIG. 1 is formed in one preferred embodiment of the method of manufacture;

FIG. 9 is a perspective view of a deployed stent configured in accordance with the invention having only a frame formed from a single loop of wire formed into a series of transverse arcuate sections and longitudinal connecting sections;

FIG. 10 is a perspective view of a stent in a deployed state and configured in accordance with another embodiment of the invention, having first and second half-frames, each formed from a piece of wire formed into a series of arcuate sections and longitudinal connecting sections and a plurality of connecting segments connecting opposed longitudinal connecting sections on both sides of the stent;

FIG. 11 is a plan view of the deployed stent of FIG. 10;

FIG. 12 is a side view of the deployed stent of FIG. 10;

FIG. 13 is a perspective view of an alternate configuration of the stent of FIG. 10 in which connecting segments are present on only one side of the stent;

FIG. 14 is a plan view of the stent of FIG. 10 is a compressed, predeployed state;

FIG. 15 is a side view of the stent of FIG. 10 is a compressed, predeployed state;

FIG. 16 is a perspective view of a stent in a deployed state, configured in accordance with another embodiment of the invention, having opposed arcuate sections, opposed longitudinal connecting sections and connecting segments or hinges on both sides and formed from a laser cut piece of hypotubing;

FIG. 17 is a plan view of the deployed stent of FIG. 16;

FIG. 18 is a side view of the deployed stent of FIG. 16;

FIG. 19 is a plan view of a stent in a deployed state, configured in accordance with another embodiment of the invention, having longitudinally offset arcuate loop sections, and connecting segments or hinges only on one side and formed from a laser cut piece of hypotubing;

FIG. 20 is a side view of the stent of FIG. 19;

FIG. 21 is a rolled out detail of the stent of FIGS. 19 and 20;

FIG. 22 is a cross section of a vessel with the stent of FIG. 10 deployed in the vicinity of an aneurysm; and

FIG. 23 is a cross section of a vessel with the stent of FIG. 13 deployed in the vicinity of an aneurysm.

### DETAILED DESCRIPTION OF THE PREFERRED EMBODIMENTS

As shown in the exemplary drawings, which are provided for the purposes of illustration and not by way of limitation, the device of the present invention is designed to be deployed intravascularly without the necessity of balloons or other expansive elements and can be deployed from a guiding catheter directly into the area to be treated. The intravascular device of the present invention is particularly useful for treatment of damaged arteries incorporating aneurysms and the like, particularly those which are treatable by the use of embolic coils or other embolic devices or agents used to occlude the aneurysm. More particularly, the device of the invention is particularly well adapted to use with the types of catheters used to place such embolic coils in aneurysms, and the device may be used to reinforce the area in the vicinity of the aneurysm while allowing placement of one or more embolic coils through the gaps in the device, while assisting in the retention of the embolic devices within the dome of the aneurysm.

In general, device of the invention is formed of superelastic or shape memory material, which, in its deployed configuration comprises a series of opposed arcuate sections connected by opposed longitudinal sections. The opposed arcuate sections from a series of or circumferential loops. Upon deployment, the device is placed within the vasculature so that it extends from a position proximal to a position distal of the aneurysm to be treated. The device may be arranged so that an open portion of the device straddles the neck of the aneurysm to allow placement of embolic coils and the like through the opening into the aneurysm.

In one configuration of the device, placement near the aneurysm is achieved by deforming the device into a flattened and compressed state and positioning it within a guiding catheter. Once the guiding catheter is placed near the aneurysm, the device is pushed out of the guiding catheter by means of a pusher and detached from the pusher by a variety of means to complete placement of the device. After placement of the device, the pusher and catheter are withdrawn.

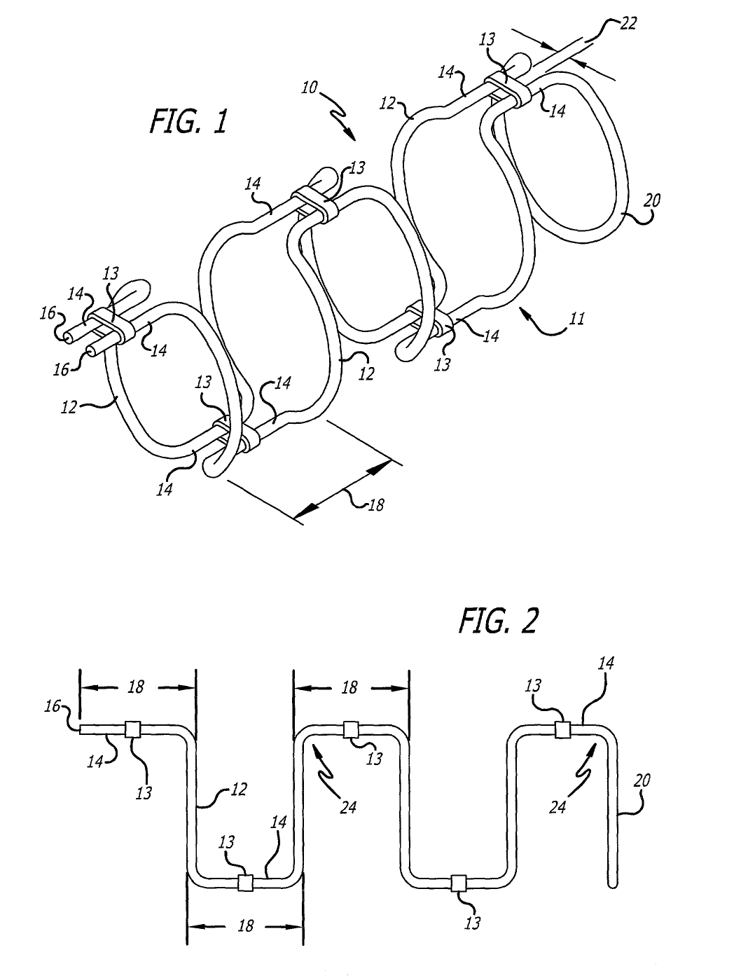

Turning now to the drawings, in which like reference numerals are used to designate like or corresponding elements among the several figures, in FIG. 1, there is shown one embodiment of an intravascular device 10, i.e., stent, for use in vasoocclusive procedures. The stent 10 includes a frame 11 and a plurality of connecting segments 13 connecting portions of the frame.

With reference to FIGS. 1 and 2, in one configuration of the stent 10, the frame 11 is formed from a single piece of wire configured as a series of arcuate sections 12 connected by longitudinal sections 14 to progressively form an essentially cylindrical frame. More specifically, the two free ends 16 of the piece of wire are placed in close proximity to each other. A first pair of longitudinal sections 14 extends from the free ends 16. The wire is then formed into a pair of arcuate sections 12 extending in semi-circular arcs for a distance less than half of the circumference of the frame to a position in which a transition into a second pair of longitudinal sections 14 are formed for a second distance 18 at which point they transition back to another pair of arcuate sections 12 and then proceed in such a sequence towards a continuous end loop 20 extending between the most distal longitudinal sections 14 to form the distal end of the frame. The distance 18 between adjacent arcuate sections 12 is selected such that the space between adjacent loops is sufficient to allow for the passage of an embolic device. The transition 24 between the arcuate sections 12 and the longitudinal sections 14 have a predetermined radius.

In one embodiment, the wire of the frame 11 is made of a superelastic material such as a nickel-titanium alloy to allow for easy insertion of the stent 10 within a guiding catheter. The wire may be coated with a corrosion resistant material such as Parylene. Other materials, such as shape-memory alloys, may also be used to provide for the dual purposes of ease of insertion into a guiding catheter and formation upon deployment into the desired shape of the device. One material that is contemplated as a wire from which the frame 11 can be made is a stranded cable including one or more radiopaque strands, or which has radiopaque markers deployed along its length. Such a stranded cable can be made of a variety of materials including stainless steel, shape-memory alloy, superelastic alloy, platinum or the like or combinations thereof. While this configuration of the frame 11 is shown in the form of a cylindrical wire, those skilled in the art will realize that other configurations of material may be used to form the frame, including laminates, flatten wires and laser cut hypotubing, each of which are within the scope of the invention.

With continued reference to FIG. 1, the frame 11 is configured such that the longitudinal sections 14 are arranged in opposed pairs. In accordance with the invention, one or more connecting segments 13 span the gap 22 between opposed longitudinal sections 14 to thereby connect the sections. The connecting segments 13 may be on both sides of the frame or alternatively (not shown) on only one side of the frame.

In one embodiment, the connecting segments 13 are bands wrapped around opposed longitudinal sections 14. The band 13 may be made of a plastic material, such as polytetrafluoroethylene (PTFE) or a metallic material, such as platinum or stainless steel. The ends of the bands 13 are secured together through bonding, crimping or soldering, depending on the specific band material. A radiopaque material may be included in the connecting segments 13 to aid visibility.

In another configuration (not shown), the connecting segments 13 include two individual bands, one wrapped around each of the opposed longitudinal sections 14. These bands are then secured to each other by bonding or soldering. In yet another configuration (also not shown), the connecting segments 13 may be a piece of solder spanning the gap 22 between the opposed longitudinal sections 14.

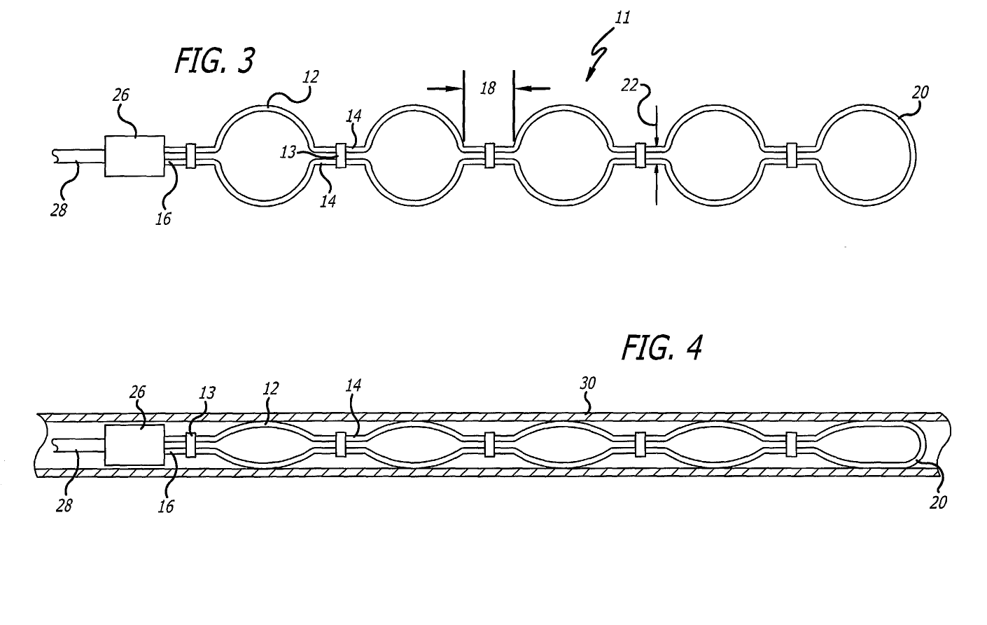

With reference to FIG. 3, the stent 10, prior to deployment in a vessel, can be made into an essentially flat configuration in which the free ends 16 of the stent are connected to a deployment device 26 on the distal end of a pusher 28 which fits within a guiding catheter (not shown). In this configuration, it can be seen that the arcuate sections 12 are connected by the longitudinal sections 14 which become essentially parallel with the longitudinal axis of the stent in the deployed configuration. The connecting segments 13 connecting the longitudinal sections 14 maintain the opposite sides of the frame 11 generally fixed relative to each other and thereby provide increased stability along the length of the stent. This increased stability reduces the possibility of the stent 10 bending or kinking during placement of the stent in the guiding catheter and subsequent deployment.

With reference to FIG. 4, prior to placement within a vessel, the stent 10 is placed within a guiding catheter 30 by first attaching the stent to the deployment device 26 on the pusher 28 and then pulling the stent into the guiding catheter using the pusher. During this process the arcuate sections 12 of the flattened stent 10 become compressed. In this state the stent 10 looks like a plurality of stretched linear loops of wire connected in series. The guiding catheter 30 is then introduced into the vasculature and positioned near the area of the vasculature to be treated. Once positioned, the pusher 28 is pushed in the distal direction to extend the stent 10 from the guiding catheter 30.

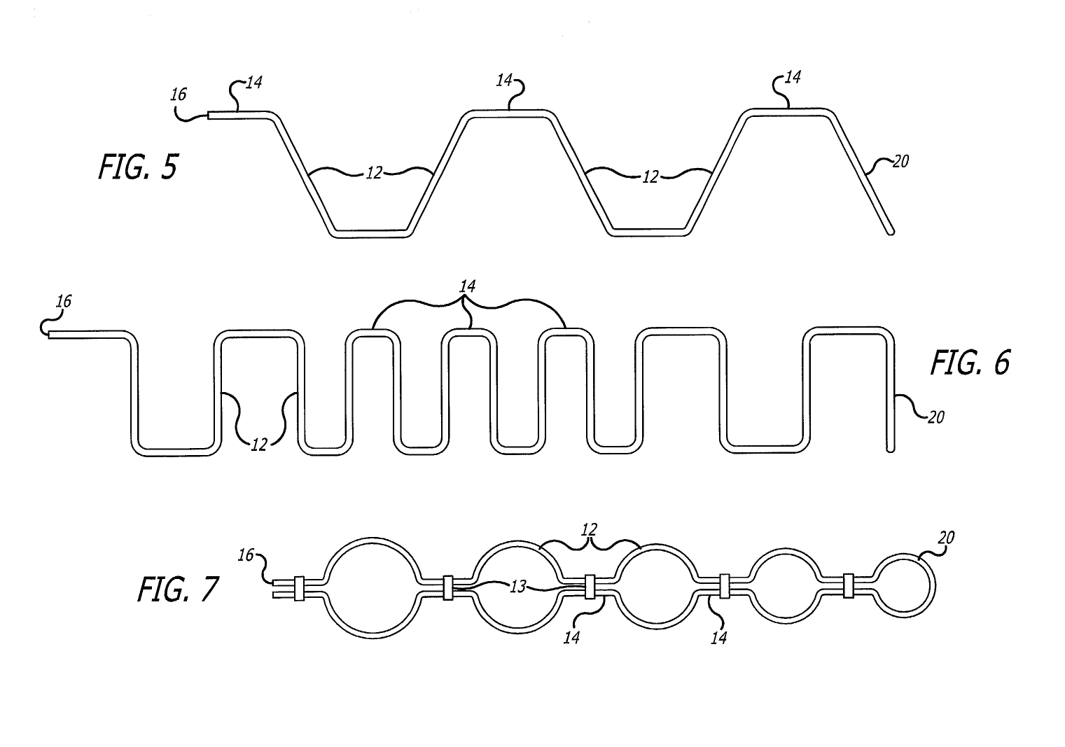

With reference to FIG. 5, as the stent 10 is deployed from the guiding catheter (not shown) the compressed arcuate sections 12 begin to assume their normally arcuate shape while the frame 11 itself begins to assume its cylindrical shape. Eventually, the stent 10 returns to the shape as shown in FIG. 1. During this process, the detachment device 26 separates from the ends 16 of the stent 10 and is withdrawn into the catheter 30 (FIG. 4) and removed from the vasculature.

The frame 11 portion of the stent 10 maybe formed in various different configurations. For example, in one configuration the density of arcuate sections can be varied from proximal to distal end in order to provide a relatively greater density in an area to be placed in a portion of the vasculature which is particularly weak or is threatened by treatment. With reference to FIG. 6, in one such configuration the stent 10 can be formed to have shorter longitudinal sections 14 between the arcuate sections 12 at certain sections of the stent, for example, the middle region, and thus provide a higher degree of reinforcement in that specific area. Such a configuration has numerous benefits depending on the topology of the damage to the artery, and can provide benefits for certain types of treatment therapies.

As another example, the stent may be configured to have a variable diameter in the arcuate sections over the length of the stent in order to provide relatively greater circumferential tension against the wall of the vessel in some areas than others. With reference to FIG. 7, in one such configuration the stent 10 may be formed such that the radii of the arcuate sections 12 vary along the length of the stent. In FIG. 7, the radii progressively decrease in size from the proximal end to the distal end of the stent. Other arrangement are possible. For example, the radii may taper down in size from both ends of the stent toward the middle. Any of the preceding configurations allow the stent to modify the blood flow characteristics in the vessel in which the stent is deployed. In another configuration (not shown), the arcuate sections are formed into an arcuate curve having a radius that varies over the length of the loop.

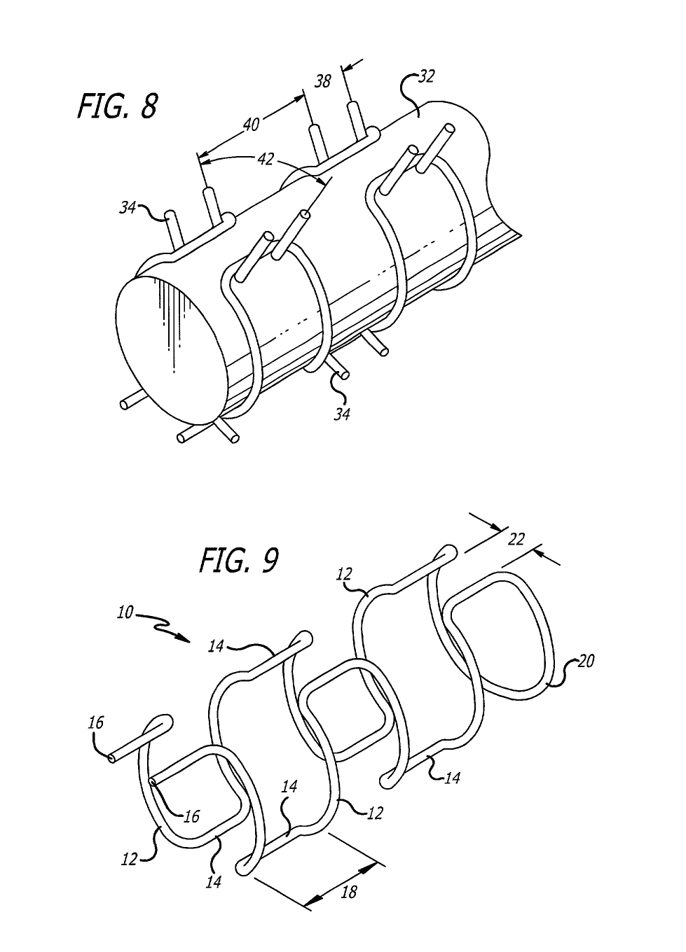

This configuration of the stent may be formed in a number of ways, but there are presently two preferred methods of manufacture. In a first preferred method illustrated in FIG. 8, a longitudinal mandrel 32 made of tungsten, ceramic, stainless steel or other heat resistant material has inserted into it pegs 34 of heat resistant material around which the wire to be formed into the frame is wound. The position of the pegs represent transitions between the arcuate sections 12 and the longitudinal sections 14 of the frame. The diameter of the pegs 36 and the spacing of the pegs 38, 40, 42 may be altered in order to provide certain characteristics that are desired in the stent as it is formed. Alternatively, the mandrel can have a grooved configuration formed into it in which the wire is placed prior to heat treatment.

In either method, a single wire is wound progressively down the mandrel forming arcuate sections 12 and longitudinal sections 14 until a desired length of the stent is reached, at which point the path is retraced similarly to the position at which the frame was begun on the mandrel. The wire can then be heat treated on the mandrel to create a shape memory or treated to reach a superelastic state.

After formation, the frame 11 is removed from the mandrel 32 and one or more connecting segments 13 are secured to opposing longitudinal sections 14. The connecting segments 13 are secured to the longitudinal sections 14 using bonding or soldering processes well known to those skilled in the art. Thereafter, the stent can be stretched to be inserted into a guiding catheter prior to insertion into the vasculature or compressed over tubing and constrained in a sheath.

As previously mentioned with reference to FIGS. 6 and 7, the stent can be formed in a variety of configurations. In other such configuration overlapping of the arcuate sections 12 and the longitudinal sections 14 create particularly desired characteristics to the stent and thereby enhance specific aspects of density or longitudinal pushability for various applications.

In another configuration, as shown in FIG. 9, the stent 10 is formed of a single loop of superelastic or shaped-memory wire shaped into a series of transverse loops and longitudinal connecting sections similar to the previously described stent shown in FIG. 1. This configuration, however, does not include the connecting segments 13 (FIG. 1) as in the previous stent. It has been noted, however, that due to its single loop configuration this stent may bend and kink along its length while being pulled into or pushed from the catheter. Such bending and kinking may damage the structural integrity of the stent. Once deployed, the stent assumes its expanded state and provides reinforcement to the vessel wall. In this regard, the single loop configuration may not provide sufficient radial support due to the gaps 22 between opposing sides of the stent. For these reasons the stent shown in FIG. 1 is a preferred embodiment.

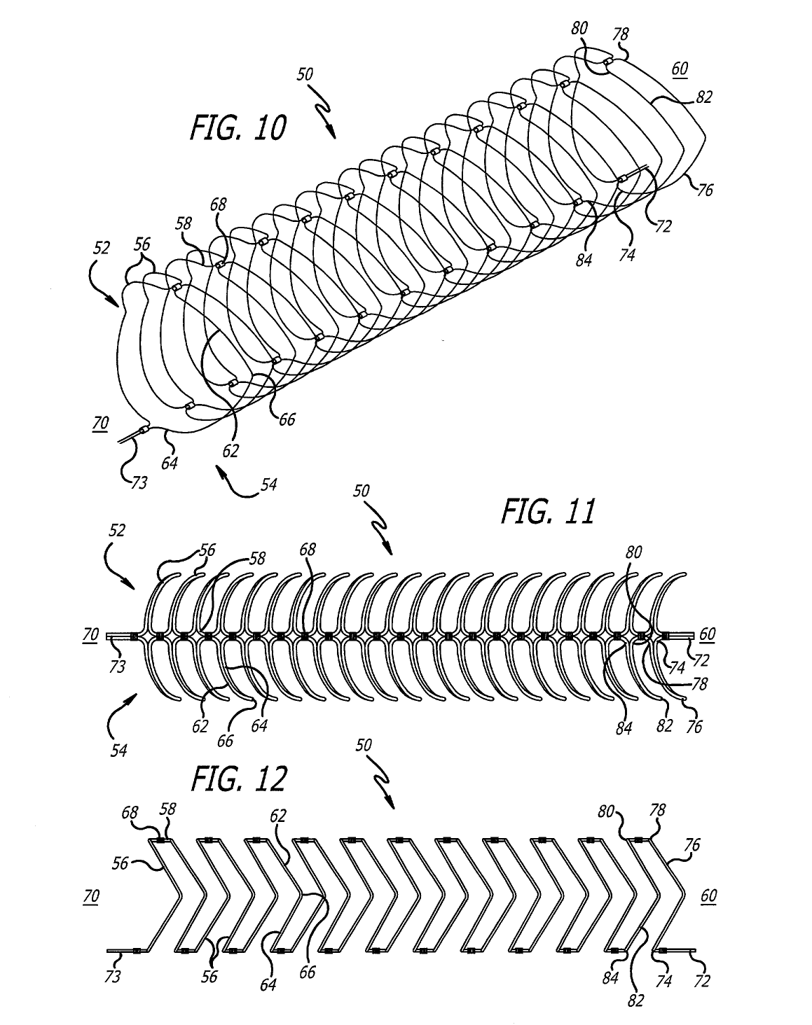

With reference to FIGS. 10, 11 and 12, in another embodiment of the invention, a stent 50 is formed to include a first half-frame 52 and a second half-frame 54. Each of the half-frames 52, 54 include a plurality of generally parallel arcuate sections 56 connected by longitudinal sections 58. In this embodiment, the longitudinal sections 58 are not linear as in the previous embodiment but instead are curved. The arcuate sections 56 are generally semicircular in shape when viewed along the axis of the stent, bow toward the proximal end 60 of the stent when viewed from the top (FIG. 11) and have a chevron configuration, with top and bottom portions 62, 64 meeting at an angle 66 pointing toward the proximal end 60, when viewed from the side (FIG. 12).

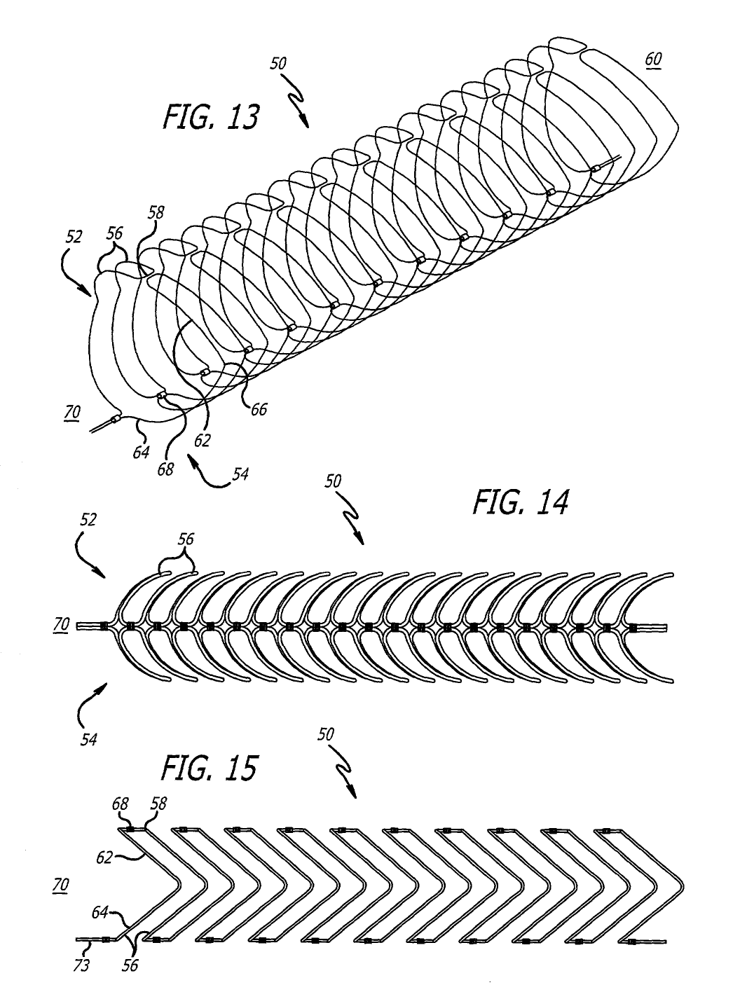

The stent 50 also includes a plurality of connecting segments 68. These segments 68 may be a single band, a pair of bands or solder, as previously described with reference to the stent configuration shown in FIG. 1. The connecting segments 68 secure a plurality of first half-frame longitudinal sections 58 to a plurality of second half-frame longitudinal sections 58 such that the first half-frame and the second half-frame form a cylinder. In the configuration of FIG. 10, the connecting segments 68 are on both sides of the cylinder. As such the stent has improved radial strength. In an alternate configuration, as shown in FIG. 13, the connecting segments 60 are only located on one side of the cylinder. As such the stent has improved collapsing capacity which is beneficial during stent deployment.

With continued reference to FIGS. 10 and 13, each of the first and second half-frames 52, 54 are formed from a separate piece of elongate resilient wire. In one embodiment, the wire is made of a superelastic material such as a nickel-titanium alloy to allow for easy insertion of the stent 50 into a guiding catheter or sheath. The wire may have either a circular or flatten cross section and maybe coated with a corrosion resistant material such as Parylene. Other materials, such as shape-memory alloys, may also be used. One material that is contemplated as a wire from which the half-frames 52, 54 can be made is a stranded cable including one or more radiopaque strands, or which has radiopaque markers deployed along its length. Such a stranded cable can be made of a variety of materials including stainless steel, shape-memory alloy, superelastic alloy, platinum or the like or combinations thereof.

Each piece of wire has a first end 72 extending distally from the proximal end 60 of the half frame. After a predetermined distance, the wire transitions at a first point 74 to a first arcuate section 76 and then transitions to a first longitudinal section 78 for a length to a second point 80. The piece of wire then transitions to a second arcuate section 82 and a second longitudinal section 84 and proceeds similarly to its second end 73 at the distal end 70 of the half-frame. The first end 72 and the second end 73 of the first half-frame 52 and second half-frame 54 may be secured together by a connecting segment 68. Alternatively, the ends 72, 73 may be left free.

The resilience of the wire from which the half-frames are formed allows for the frames to transition between a predeployed essentially flat configuration, similar to that shown in FIG. 3, and a deployed generally cylindrical configuration, as shown in FIG. 10. This allows for placement of the stent in a guiding catheter as previously described.

The resilience of the wire, in combination with the bow and chevron configuration, also allows for the half-frames 52, 54 to transition between a predeployed radially compressed configuration, as shown in FIGS. 14 and 15, and a deployed generally cylindrical configuration, as shown in FIGS. 10 and 13. With reference to FIG. 14, when radially inward pressure is applied to the sides of the stent, the bowed portions of the adjacent arcuate sections 56 collapse toward each other. Similarly, with reference to FIG. 15, when radially inward pressure is applied to the top and the bottom of the stent, the top portion 62 and bottom portion 64 of the arcuate sections 56 collapse toward each other. Accordingly, when the stent experiences each of top, bottom and side radially inward pressure the stent reduces in size radially. The reduction in radial size allows for placement of the stent in a guiding catheter or sheath without having to flatten and stretch the stent as previously described.

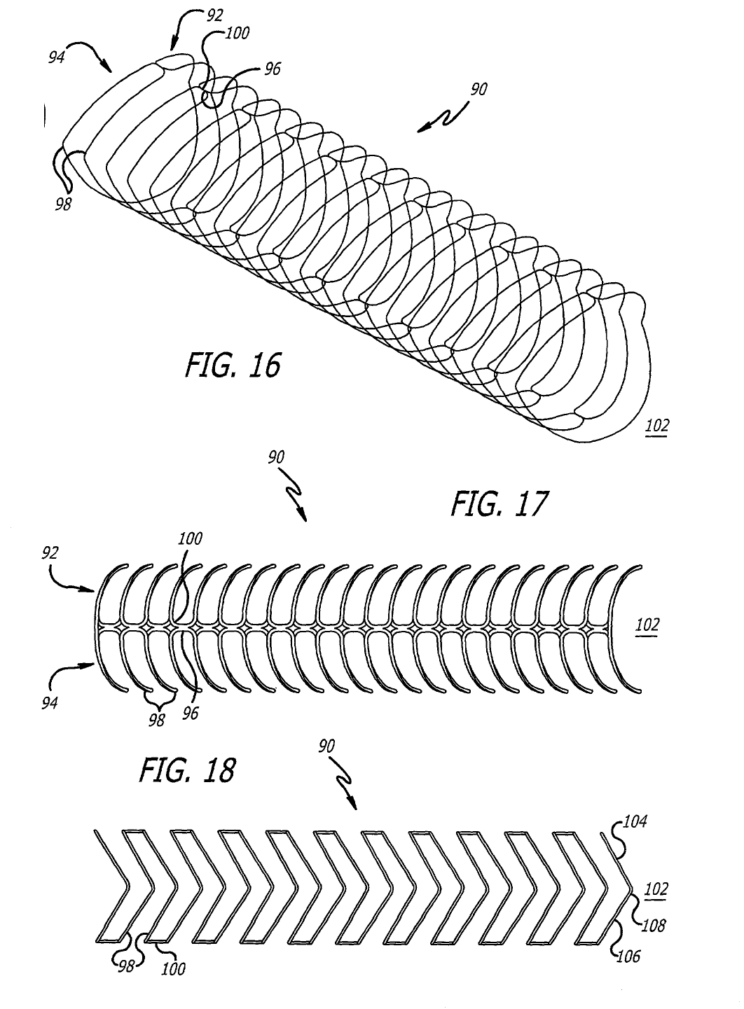

With reference to FIGS. 16, 17 and 18, in another embodiment of the invention, a stent 90 is formed by laser cutting a piece of hypotubing to form a stent pattern including a first half-frame 92, a second half-frame 94 and a plurality of connecting segments 96. The hypotubing may be formed from a shape-memory material similar to that of the resilient wire of the previous configuration. Since the stent is laser cut from a piece of hypotubing there are no discreet parts such as the described first half-frame 92, second half-frame 94 and plurality of connecting segments 96. However, for description purposes these various parts are referred to herein.

The first and second half-frames 92, 94 are each patterned to respectively include a plurality of generally parallel arcuate sections 98 connected by longitudinal sections 100. The arcuate sections 98 are generally semicircular in shape when viewed along the axis of the stent, bow toward the proximal end 102 of the stent when viewed from the top (FIG. 17) and have a chevron configuration, with top and bottom portions 104, 106 meeting at an angle 108 pointing toward the proximal end 102, when viewed from the side (FIG. 18). Opposed longitudinal sections 100 are joined by connecting segments 96 or hinges.

In the configuration of FIG. 16, the connecting segments 96 are on both sides of the cylinder. As such the stent has improved radial strength. In another configuration (not shown), the stent maybe formed such that the connecting segments 96 are only located on one side of the stent. As such the stent has improved collapsing capacity which is beneficial during stent deployment. In either configuration, the stent 90 is formed from hypotubing having resiliency characteristics like that of the wire stent configurations (FIGS. 1 and 10). Accordingly, it may be flattened and stretched or radially compressed for placement in a guiding catheter or sheath.

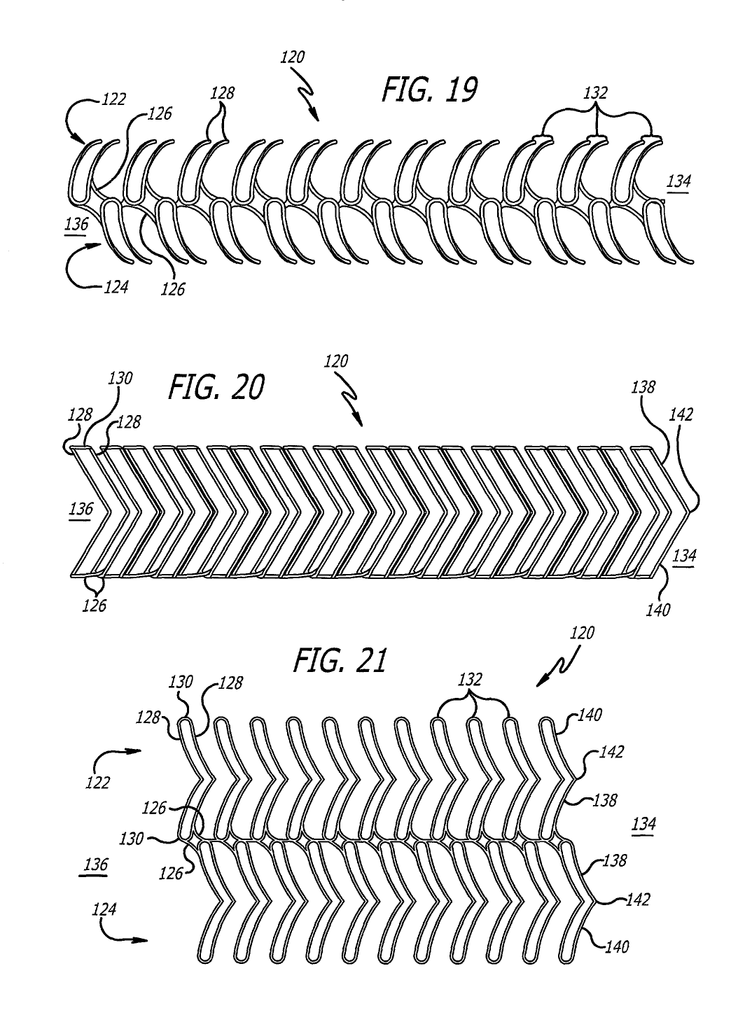

With reference to FIGS. 19, 20 and 21, in another embodiment of the invention, the stent 120 is formed by laser cutting a piece of hypotubing to form a stent pattern having a first half-frame 122, a second half-frame 124 and a plurality of connecting segments 126. The first and second half-frames 122, 124 are each patterned to include a series of generally parallel arcuate loop sections 132. Each arcuate loop section 132 includes a pair of generally parallel arcuate sections 128 connected by longitudinal sections 130. The arcuate sections 128 are generally semicircular in shape when viewed along the axis of the stent, bow toward the proximal end 134 of the stent when viewed from the top (FIG. 19) and have a chevron configuration, with top and bottom portions 138, 140 meeting at an angle 142 pointing toward the proximal end 134 of the stent, when viewed from the side (FIG. 20).

Opposed acuate loop sections 132 are joined by connecting segments 126 or hinges. As with other configurations, the connecting segments 126 may be on only one side of the stent or on both sides (not shown). In a preferred embodiment, the half-frames 122, 124 are aligned relative to each other such that opposing arcuate loop sections 132 are longitudinally offset from each other, in a staggered pattern. Due to the formation of independent arcuate loop sections 132, this configuration of the stent may not be longitudinally stretched. The combination chevron and bow configuration does, however, allow for it to be radially compressed for delivery.

The invention provides numerous important advantages in the treatment of vascular malformations, and particularly malformations which include the presence of aneurysms. Since the stents do not represent an essentially solid tubular member and do not require the use of a balloon or other mechanical device for deployment, they are capable of deployment from a guiding catheter which need not occlude the artery as it is put into a position from which to deploy the stent. Furthermore, the stents upon deployment can reinforce the artery without occluding access to the aneurysm, thus allowing the stents to be deployed prior to the placement of embolic coils or the like in the aneurysms. Alternatively, depending on the nature of the vascular defect, the embolic coils or other embolic occlusive or other vasoocclusive devices can be placed and the stents deployed thereafter to hold the devices in the aneurysm.

The present invention also contains numerous advantages over the prior art, including enhanced pushability without creating circumferential stress from the loop section, as is often found in the case of coil-type intravascular flow modifiers known in the prior art. The reinforcement strength of the stents is enhanced by the connecting segments spanning opposed sections of the frames. The characteristics of the stent, such as loop strength, and the resilience of the stent are controlled by several factors including the radii of the transitions to the longitudinal sections, the diameter or thickness of the wire or hypotubing and the distance between the longitudinal sections and the arcuate sections which form the frame.

The collapsibility of the stent for deployment purposes is a function of material and stent configuration. The use of superelastic and/or shape-memory material in combination with the unique interconnection between arcuate sections allows for the stent to be flattened and stretched for placement within a guiding catheter. The addition of chevron configured arcuate sections allows for the stent to be compressed while the use of bowed arcuate sections allows for further compression and ease of movement in the distal direction during deployment. Thus, the invention provides a wide variety of performance characteristics that can be designed as part of the stent configuration.

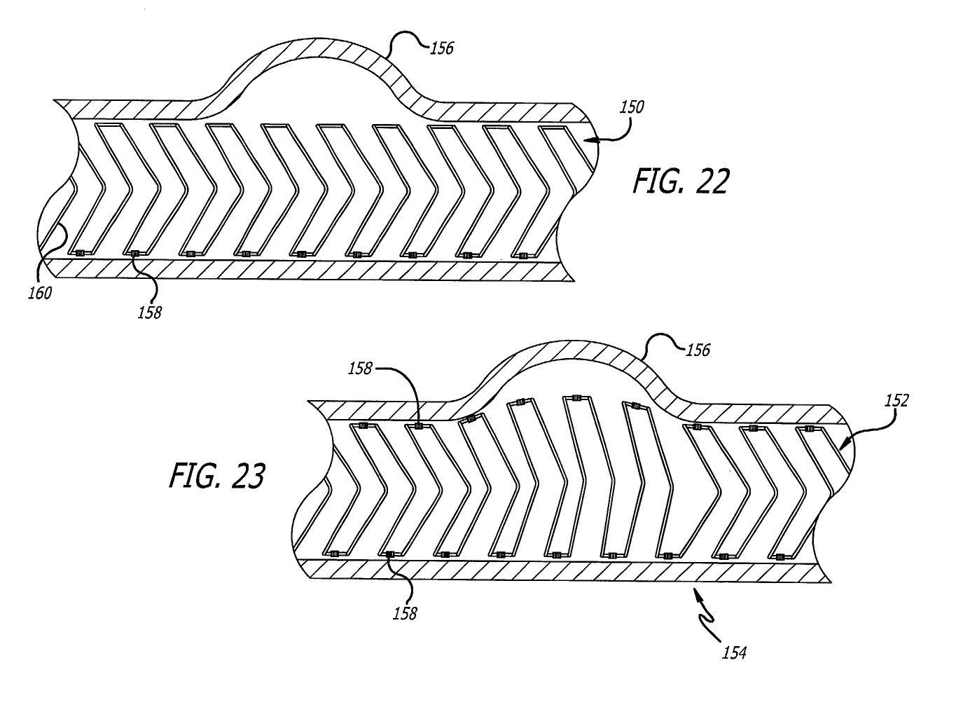

With reference to FIGS. 22 and 23, two configurations of stents 150, 152 are shown deployed within a vessel 154 in the vicinity of an aneurysm 156. The stent 150 in FIG. 22 is configured like the stent shown and described with respect to FIG. 13. This stent 150 includes connecting segments 158 on only one side of the stent. As shown, the chevron configuration of the arcuate sections 160 cause the stent to expand and fit tightly against the interior wall of the vessel. With respect to the free side of the stent, i.e., the side of the stent without connecting segments 158, it has been noted that the disconnect between the opposed arcuate sections decreases the radial strength of the stent on that side and makes the stent more compliant. This compliance allows the stent to expand to a generally uniform diameter along its length without entering into the area of the aneurysm 156. Thus the stent 150 provides support for the vessel 154 in the area around the aneurysm 156 while leaving room for the introduction of embolic coils into the aneurysm.

The stent 152 in FIG. 23 is configured like the stent shown and described with respect to FIG. 10. This stent 150 includes connecting segments 158 on both sides of the stent. As a result, the stent has increased radial strength on both sides, is less compliant than the stent shown in FIG. 22 and thus tends to expand into a portion of the area of the aneurysm 156.

From the above, it may be observed that the present invention provides significant benefits to the treatment of vascular malformations, and particularly aneurysms in the neurovasculature. Importantly, the invention is particularly advantageous when used in combination with vasoocclusive devices placed in the aneurysm by intravascular procedures. The stents of the present invention may also find application in the treatment of ischemic diseases.

It will be apparent from the foregoing that while particular forms of the invention have been illustrated and described, various modifications can be made without departing from the spirit and scope of the invention. Accordingly, it is not intended that the invention be limited, except as by the appended claims.

What is claimed is:

1. A stent for use in the intravascular treatment of blood vessels, comprising:
a first half-frame and a second half-frame, each comprising a plurality of arcuate sections connected by longitudinal sections; and
a plurality of connecting segments securing a plurality of first half-frame longitudinal sections to a plurality of second half-frame longitudinal sections such that the first half-frame and the second half-frame form a cylinder.

2. The stent of claim 1 wherein the arcuate sections have a chevron configuration.

3. The stent of claim 2 wherein the point of the chevron is directed toward the proximal end of the stent.

4. The stent of claim 1 wherein the arcuate sections have a bowed configuration.

5. The stent of claim 4 wherein the arcuate sections bow toward the proximal end of the stent.

6. The stent of claim 1 wherein connecting segments are located on only one side of the cylinder.

7. The stent of claim 1 wherein connecting segments are located on both sides of the cylinder.

8. The stent of claim 1 wherein each of the first and second half-frames are formed from a piece of elongate resilient wire with a first end extending distally from the proximal end of the half frame, thereafter transitioning at a first point to a first arcuate section, thereafter transitioning to a first longitudinal section for a length to a second point, thereafter transitioning to a second arcuate section and a second longitudinal section and proceeding similarly to the distal end of the half frame.

9. The stent of claim 8 wherein each of the half frames has a predeployed essentially flat configuration and a deployed generally cylindrical configuration.

10. The stent of claim 8 wherein each of the half frames has a predeployed radially compressed cylindrical configuration and a deployed generally cylindrical configuration.

11. The stent of claim 8 wherein the resilient wire has an essentially flat cross section.

12. The stent of claim 8 wherein the connecting segments comprise:
a first band around one of the first half-frame longitudinal sections; and
a second band around one of the second half-frame longitudinal sections;
wherein the first and second bands are secured together.

13. The stent of claim 12 wherein the first and second bands are metallic and are secured together by solder.

14. The stent of claim 8 wherein the connecting segments comprise a band secured around one of the first half-frame longitudinal sections and one of the second half-frame longitudinal sections.

15. The stent of claim 8 wherein the connecting segments comprise a piece of solder.

16. The stent of claim 1 wherein the first and second half-frames and the connecting segments are formed from a single piece of hypotubing with portions removed to form:
first and second half-frame patterns, each having a first end extending distally from the proximal end of the half frame, thereafter transitioning at a first point to a first arcuate section, thereafter transitioning to a first longitudinal section for a length to a second point, thereafter transitioning to a second arcuate section and a second longitudinal section and proceeding similarly to the distal end of the half frame; and
the plurality of connecting segments.

17. The stent of claim 16 wherein each of the half frames has a predeployed essentially flat configuration and a deployed generally cylindrical configuration.

18. The stent of claim 16 wherein each of the half frames has a predeployed radially compressed configuration and a deployed generally cylindrical configuration.

19. A stent for use in the intravascular treatment of blood vessels, comprising:
a first half-frame and a second half-frame, each comprising a plurality of arcuate loop sections which comprise a pair of arcuate sections connected at each end by a longitudinal connecting section; and
a plurality of connecting segments securing a plurality of first half-frame arcuate loop sections to a plurality of second half-frame arcuate loop sections such that the first half-frame and the second half-frame form a cylinder.

20. The stent of claim 19 wherein the first and second half-frames are formed from a material having properties that provide it with a predeployed radially compressed configuration and a deployed generally cylindrical configuration.

21. The stent of claim 19 wherein the arcuate sections have a chevron configuration.

22. The stent of claim 21 wherein the point of the chevron is directed toward the proximal end of the stent.

23. The stent of claim 19 wherein the arcuate sections have a bowed configuration.

24. The stent of claim 23 wherein the arcuate sections bow toward the proximal end of the stent.

25. The stent of claim 19 wherein connecting segments are located on only one side of the cylinder.

26. The stent of claim 19 wherein connecting segments are located on both sides of the cylinder.

27. The stent of claim 19 wherein the first and second half-frame arcuate loop sections are secured such that the first half-frame arcuate loop sections are longitudinally offset from the second half-frame arcuate loop sections.

28. A stent for use in the intravascular treatment of blood vessels, comprising:
a generally cylindrical frame formed of an elongate resilient wire, the two free ends of the wire extending distally from the proximal end of the frame, thereafter transitioning at a first point to a pair of opposed first arcuate sections, thereafter transitioning to a pair of opposed first longitudinal sections for a length to a second point, thereafter transitioning to a pair of opposed second arcuate sections and a pair of opposed second longitudinal sections and proceeding in a like pattern to the distal end of the frame; and
a plurality of connecting segments, connecting a plurality of opposed longitudinal sections.

29. The stent of claim 28 wherein the frame is formed from a material having properties that provide it with a predeployed essentially flat configuration and a deployed generally cylindrical configuration.

30. The stent of claim 28 wherein connecting segments are located on both sides of the frame.

31. The stent of claim 28 wherein connecting segments are located on only one side of the frame.

32. The stent of claim 28 wherein the connecting segments comprise a pair of bands, one around each of opposed longitudinal sections, wherein the first and second bands are secured together.

33. The stent of claim 32 wherein the bands are metallic and are secured together by solder.

34. The stent of claim 32 wherein the bands are plastic and are secured together by bonding material.

35. The stent of claim 28 wherein the connecting segments comprise a single band secured around both of an opposed pair of longitudinal sections.

36. The stent of claim 28 wherein the connecting segments comprise a piece of solder spanning between a pair of opposed longitudinal sections.

37. The stent of claim 28 wherein the connecting segments comprise a radiopaque material.

38. The stent of claim 28 wherein the free ends of the frame are attached to deployment means at the distal end of a pusher for deploying the frame in the vasculature of a patient.

39. The stent of claim 28 wherein the arcuate sections are spaced apart distally along the frame by a predetermined distance sufficient to allow passage of an embolic coil between the adjacent sections.

40. The stent of claim 28 wherein the distal end of the stent comprises a continuous loop extending between the most distal longitudinal sections.
